(19) United States
(12) Patent Application Publication
(10) Pub. No.: US 2008/0096939 A1
(43) Pub. Date: Apr. 24, 2008

(54) Process For Preparation Of Pramipexole By Chiral Chromatography

(76) Inventors: Andreas Keil, Darmstadt (DE); Michael Schulte, Darmstadt (DE)

Correspondence Address: WOLF GREENFIELD & SACKS, P.C., 600 ATLANTIC AVENUE, BOSTON, MA, 02210-2206, US

(21) Appl. No.: 11/630,416

(22) PCT Filed: Jun. 29, 2005
(86) PCT No.: PCT/GB05/50098

§ 371 Date: Sep. 10, 2007

(30) Foreign Application Priority Data
Jul. 3, 2004 (GB) 0414968.8
Jul. 14, 2004 (GB) 0415721.0

(51) Int. Cl. A61K 31/428; C07D 277/82

(52) U.S. Cl. 514/367; 548/161

(57) ABSTRACT

A novel process for the preparation of S(−)-2-amino-6-propylamino-4,5,6,7-tetrahydrobenzothiazole (pramipexole).

### TECHNICAL FIELD

The present invention relates to a process for the preparation of S(−)-2-amino-6-propylamino-4,5,6,7-tetrahydrobenzothiazole (pramipexole), the process comprising chiral chromatography. The process is suitable for being performed on an industrial scale.

The present invention also relates to highly pure pramipexole, or a pharmaceutically acceptable salt thereof, which may be prepared by the chiral chromatography process of the present invention. Pramipexole and its salts may be used for the treatment of a psychiatric or neurological disorder, such as schizophrenia, Alzheimer's disease or Parkinson's disease.

### BACKGROUND ART

The present invention relates to a novel process for the preparation of S(−)-2-amino-6-propylamino-4,5,6,7-tetrahydrobenzothiazole (pramipexole).

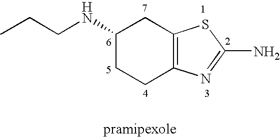

Certain 2-amino-4,5,6,7-tetrahydro-6-aminobenzothiazoles are known to have dopamine D-2 activity and are therefore potentially useful as pharmaceuticals for the treatment of psychiatric disorders such as schizophrenia and Alzheimer's disease. One such compound, the dihydrochloride salt of S(−)-2-amino-6-propylamino-4,5,6,7-tetrahydrobenzothiazole (pramipexole) is marketed as a pharmaceutical for the treatment of Parkinson's disease. Pramipexole is marketed as the S(−) enantiomer as the dopiaminergic activity of the S(−) enantiomer is twice as high as that of the corresponding R(+) enantiomer.

However, the preparation of the single enantiomer requires a more complex manufacturing process than the preparation of racemic pramipexole [R,S(±)-2-amino-6-propylamino-4,5,6,7-tetrahydrobenzothiazole].

Single enantiomer compounds are typically prepared on an industrial scale via classical resolution of a racemic mixture of the final compound or of a racemic intermediate.

More recently, techniques have improved such that the single enantiomer can be prepared via an asymmetric synthetic process which affords the single enantiomer directly with no further need for resolution of a racemic mixture. However yields of asymmetric syntheses are typically not ideal for a commercial scale manufacture and the reagents used can be expensive and not environmentally friendly.

Therefore on a commercial scale, the preparation of single enantiomer compounds usually involves a resolution step. These resolution techniques usually involve formation of diastereometic salts or derivatives and separation of the diastereomers or salts by fractional crystallisation with subsequent modification and isolation of the single enantiomer.

An example of this type of resolution process in the production of pramipexole has been disclosed by C. S. Schneider & J. Mierau in J. Med. Chem., 1987, vol. 30, pages 494-498, wherein a tartaric acid salt is used for the resolution of an intermediate compound.

However, these classical techniques are inconvenient as they can add extra steps to the process and resolution of intermediates may not ultimately lead to final compounds of very high optical purity.

Alternatively, resolution can be achieved by chromatographic separation e.g. by separation of the racemic mixture directly using a chiral stationary phase or by derivatising the racemic mixture into a diastereometic mixture and separation of the diastereomers using a standard stationary phase. The latter option further requires chemical conversion of one separated diastereomer into the required enantiomer.

However, in practice, these chromatographic resolution techniques generally fail to afford any meaningful commercial quantities of the desired pure enantiomer and are generally only used for production of small laboratory scale amounts. Processes for the preparation of racemic pramipexole are disclosed in patents EP 0186087 B1, U.S. Pat. No. 4,843,086, U.S. Pat. No. 4,886,812 and patent application WO 04/026850 A1.

A process for the preparation of racemic pramipexole and its resolution, as discussed above, is disclosed by C. S. Schneider & J. Mierau in J. Med. Chem., 1987, vol. 30, pages 494-498.

An alternative prior art disclosure for a stereoselective process for the preparation of pramipexole enriched in the desired enantiomer is contained in patent applications WO 02/22590 A1 and WO 02/22591 A1. The methods disclosed therein utilise an enantioselective reductive amination. However, the reductive amination is only stereoselective and not stereospecific and further enantiomeric purification has to be performed utilising conventional optical resolution (e.g. by fractional crystallisation of salts with an optically active acid).

Consequently, as discussed above, these known processes for the preparation of pramipexole are not particularly satisfactory for industrial scale manufacture.

Therefore there is a need for a more efficient process for the preparation of pramipexole on a manufacturing scale.

### SUMMARY OF THE INVENTION

A first aspect of the present invention provides a process for the preparation of pramipexole comprising chiral chromatography. For the purposes of the present invention, the term “pramipexole” is defined as S(−)-2-amino-6-propylamino-4,5,6,7-tetrahydrobenzothiazole.

For the purposes of the present invention, the term “chiral chromatography” is defined as chromatography using either a chiral stationary phase or a chiral mobile phase. Preferably the chiral chromatography process of the present invention is carried out using a chiral stationary phase.

Preferably the chemical purity of the pramipexole produced is 99% or more as measured by HPLC, more preferably 99.5% or more, even more preferably 99.83% or more.

Preferably the optical purity of the pramipexole produced is ≧96%, more preferably ≧98%, more preferably ≧99%, even more preferably ≧99.42%. For the purposes of the present invention, the term “optical purity” is defined as the percentage of a given enantiomer in an enantiomeric mixture when measured by chiral HPLC.

Preferably the optical rotation of the pramipexole produced is −88.7° (c=1, EtOH) or lower. For the purposes of the present invention, the term “−88.7° or lower”

includes −89°, −90°, −91°, −92°, −93°, and so on. The optical rotation is measured at 20° C.

Preferably the optical rotation of pramipexole dihydrochloride produced is −67.7° (c=1, MeOH) or lower. For the purposes of the present invention, the term “−67.70 or lower” includes −68°, −69°, −70°, −71°, −72°, and so on. The optical rotation is measured at 20° C.

Preferably, in the process of the present invention, racemic or enantiomerically enriched pramipexole is resolved by chiral chromatography. Preferably racemic pramipexole is resolved by chiral chromatography.

For the purposes of the present invention, the term “racemic pramipexole” is defined as a mixture of pramipexole: R(+)-2-amino-6-propylamino-4,5,6,7-tetrahydrobenzothiazole in the ratio of 55:45 to 45:55, preferably in the ratio of about 50:50. The term “enantiomerically enriched pramipexole” is defined as a mixture, wherein the percentage of pramipexole is greater than the percentage of R(+)-2-amino-6-propylamino-4,5,6,7-tetrahydrobenzothiazole. Typically “enantiomerically enriched pramipexole” is a mixture of pramipexole: R(+)-2-amino-6-propylamino-4,5,6,7-tetrahydrobenzothiazole in the ratio of 100:0 to 55:45, preferably in the ratio of 90:10 to 60:40, more preferably 90:10 to 70:30.

Preferably the process of the present invention comprises a continuous process, more preferably a multi-column continuous process. Preferably the process of the present invention comprises a simulated moving bed process.

Preferably the stationary phase used in the chiral chromatography process comprises silica gel coated with a functionalised polysaccharide. More preferably the stationary phase is Chiralpak® AS or Chiralpak® AD.

Preferably the mobile phase used in the chiral chromatography process is selected from an alcohol, another organic solvent, and mixtures thereof. More preferably the mobile phase is selected from methanol, ethanol, propanol, isopropanol, acetonitrile, and mixtures thereof. More preferably the mobile phase is selected from an acetonitrile:alcohol mixture.

Optionally the alcohol may be methanol. If the alcohol is methanol, preferably the acetonitrile:methanol ratio is between 70:30 and 90:10, preferably the ratio is about 81:19. Alternatively the alcohol may be ethanol. If the alcohol is ethanol, preferably the acetonitrile:ethanol ratio is between 80:20 and 95:05, preferably the ratio is about 90:10.

Optionally the mobile phase further comprises a co-solvent. If used, preferably the co-solvent is an alkylamine, preferably diethylamine.

For economic efficiency, the mobile phase used in the chiral chromatography process may be recycled.

Preferably the process of the present invention is performed at a temperature of 20-30° C.

Preferably the process of the present invention is performed on an industrial scale. For the purposes of the present invention, the term “industrial scale” is defined as a per day production of 1.77 kg or more of pramipexole, preferably 10 kg or more, more preferably 30.7 kg or more.

Preferably the yield of the pramipexole produced is 74% or more of the theoretical yield, more preferably the yield of the pramipexole produced is 91% or more of the theoretical yield. For the purposes of the present invention, the term “theoretical yield” is defined as the theoretical maximum yield of an enantiomer based on the quantity of the enantiomer in the starting mixture prior to the chiral chromatography process of the present invention.

A second aspect of the present invention provides pramipexole, or a pharmaceutically acceptable salt thereof, obtained by a chiral chromatography process of the first aspect of the present invention.

The second aspect of the present invention further provides pramipexole, or a pharmaceutically acceptable salt thereof, having a chemical purity of 99% or more as measured by HPLC, preferably 99.5% or mote, mote preferably 99.83% or more.

The second aspect of the present invention further provides pramipexole, or a pharmaceutically acceptable salt thereof, having an optical purity of ≧96%, preferably ≧98%, more preferably ≧99%, even more preferably ≧99.42%. For the purposes of the present invention, the term “optical purity” is defined as the percentage of a given enantiomer in an enantiomeric mixture when measured by chiral HPLC.

For the purposes of this invention, a “salt” is any acid addition salt, preferably a pharmaceutically acceptable acid addition salt, including but not limited to a hydrohalogenic acid salt such as hydrofluoric, hydrochloric, hydrobromic and hydroiodic acid salt; an inorganic acid salt such as nitric, perchloric, sulfuric and phosphoric acid salt; an organic acid salt such as a sulfonic acid salt (for example methanesulfonic, trifluoromethanesulfonic, ethanesulfonic, isethionic, benzenesulfonic, p-toluenesulfonic or camphorsulfonic acid salt), acetic, malic, fumaric, succinic, citric, tartaric, benzoic, gluconic, lactic, mandelic, mucic, pamoic, pantothenic, oxalic and maleic acid salt; and an amino acid salt such as ornithinic, glutamic and aspartic acid salt. The acid addition salt may be a mono- or di-acid addition salt. A preferred salt is a di-hydrohalogenic, di-sulphuric, di-phosphoric or di-organic acid salt. A most preferred salt is a di-hydrochloric acid salt.

The second aspect of the present invention further provides pramipexole having an optical rotation of −88.7° (c=1, EtOH) or lower. For the purposes of the present invention, the term “−88.7° or lower” includes −89°, −90°, −91°, −92°, −93°, and so on. The optical rotation is measured at 20° C.

The second aspect of the present invention further provides pramipexole dihydrochloride having an optical rotation of −67.7° (c=1, MeOH) or lower. For the purposes of the present invention, the term “−67.7° or lower” includes −68°, −69°, −70°, −71°, −72°, and so on. The optical rotation is measured at 20° C.

Preferably the pramipexole or salt thereof of the second aspect of the present invention is suitable for use as a medicament. Preferably the medicament is suitable for the treatment of a psychiatric or neurological disorder, such as schizophrenia, Alzheimer's disease or Parkinson's disease.

A third aspect of the present invention provides a pharmaceutical composition comprising the pramipexole or salt thereof of the second aspect of the present invention and a pharmaceutically acceptable carrier or diluent. Preferably the pharmaceutical composition is suitable for the treatment of a psychiatric or neurological disorder, such as schizophrenia, Alzheimer's disease or Parkinson's disease.

A fourth aspect of the present invention provides the use of the pramipexole or salt thereof of the second aspect of the present invention for the manufacture of a medicament for the treatment of a psychiatric or neurological disorder, such as schizophrenia, Alzheimer's disease or Parkinson's disease.

A fifth aspect of the present invention provides a method of treating a psychiatric or neurological disorder, such as schizophrenia, Alzheimer's disease or Parkinson's disease, comprising administering a therapeutically effective amount of the pramipexole or salt thereof of the second aspect of the present invention or a pharmaceutical composition of the third aspect of the present invention, to a subject in need of such treatment.

### DETAILED DESCRIPTION OF THE INVENTION

The inventors have found that racemic pramipexole can be resolved efficiently on a commercial scale utilising chiral chromatography. The process is high yielding and affords products of very high optical purity.

Therefore a first aspect of the current invention is a process for the preparation of pramipexole comprising chiral chromatography.

A preferred embodiment of the first aspect of the invention is that the process for the preparation of pramipexole comprises a continuous process. A preferred embodiment of the first aspect of the invention is that the process for the preparation of pramipexole comprises a multi-column continuous process or a simulated moving bed process.

Any stationary phase and mobile phase which allows effective separation can be used in the process of the invention.

The preferred stationary phases are Chiralpak® AS or Chiralpak® AD.

Typical mobile phases are alcohols, such as methanol, ethanol, propanol, isopropanol etc. or other organic liquids such as acetonitrile. The mobile phase can be a mixture of the aforementioned solvents. Co-solvents such as diethylamine can also be used in the mobile phase.

The preferred mobile phase is an acetonitrile:alcohol mixture such as acetonitrile:ethanol (90:10) acetonitrile:methanol (81:19).

The most preferred stationary phase is Chiralpak® AD.

The preferred temperature to run the process at is 20-30° C.

The use of a multi-column continuous chromatography system is the most preferred embodiment of the first aspect of the invention as it is more efficient than other systems tested (e.g. simulated moving bed or true moving bed systems).

The pramipexole prepared by the first aspect of the invention can be further converted into a pharmaceutically acceptable salt such as dihydrochloride.

Therefore, a further aspect of the invention is pramipexole and/or its pharmaceutically acceptable salts when prepared by a process according to the current invention.

### EXAMPLES

### Example 1

Racemic pramipexole was subjected to preparative chromatography using Chiralpak® AD as the stationary phase and acetonitrile:methanol (81:19) as the mobile phase. Under these conditions, the crude material has a good solubility in the mobile phase (>40 g/l) and the retention is low (K₁=1.29 & K₂=4.07) with high selectivity (3.16).

The specific productivity of the process is 2.72 kg/kg (i.e. the yield is 74% of the theoretical yield) with an eluent consumption of 250 l/kg using a multi-column continuous chromatography process for the purification of each enantiomer at an optical purity of 99%.

The solvent can be recycled with a minor loss of <0.1% on an industrial scale.

This process is very economical and yields a production of 1.77 kg of each enantiomer per day in the pilot plant.

Scaling up to industrial scale should afford 30.7 kg of each enantiomer per day.

### Example 2

Racemic pramipexole base is dissolved in acetonitrile/methanol 81:19 (v/v) at a concentration of 8 g/l, stirred for 6 hours, filtered and connected to simulating moving bed (SMB) equipment (argon purging). After separation the solvent is removed (rotary evaporator).

The SMB equipment used is a NOVASEP Licosep Lab—stationary phase:

Chiralpak® AD 20, 8 columns NW 25×120 with 280 g stationary phase; temperature during separation: 25° C.; pressure: 35 bar; eluent consumption: 5.3 1/hour; feed: 2.33 1/hour; target: 4.4 1/hour =106 1/24 hours; separation of 450 g/24 hours.

Yield: 114 g (45.6%) (i.e. 91% of the theoretical yield)

Optical purity: 99.42%

Chemical purity (by HPLC): 99.83%

Optical rotation: [α]²⁰_{D} −88.70 to −89.3° (c=1, EtOH) Pramipexole thus obtained was converted into the dihydrochlotide salt, which was found to have an optical rotation of: [α]²⁰_{D} −67.7° (c=1, MeOH).

The optically purest pramipexole dihydrochloride disclosed in the prior art (C. S. Schneider & J. Mierau in J. Med. Chem., 1987, vol. 30, pages 494-498) was reported to have an optical rotation of [α]²⁰_{D} −67.2° (c=1, MeOH).

1. A process for the preparation of pramipexole comprising chiral chromatography.

2. A process as claimed in claim 1, wherein:
(a) the chemical purity of the pramipexole produced is 99%, 99.5%, 99.83% or more as measured by HPLC;
(b) the optical purity of the pramipexole produced is ≧96%, ≧98%, ≧99%, or ≧99.42%; and/or
(c) the optical rotation of the pramipexole produced is −88.7° (c=1, EtOH) or lower.

3.-9. (canceled)

10. A process as claimed in claim 1, wherein the pramipexole is further converted into the dihydrochloride salt having an optical rotation of −67.7° (c=1, MeOH) or lower.

11. A process as claimed in claim 1, wherein racemic or enantiomerically enriched pramipexole is resolved by chiral chromatography.

12. A process as claimed in claim 11, wherein racemic pramipexole is resolved by chiral chromatography.

13. A process as claimed in claim 1, comprising a continuous process.

14. A process as claimed in claim 13, comprising a multi-column continuous process.

15. A process as claimed in claim 1, comprising a simulated moving bed process.

16. A process as claimed in claim 1, wherein the stationary phase used in the chiral chromatography process comprises silica gel coated with a functionalized polysaccharide.

17. A process as claimed in claim 16, wherein the stationary phase is Chiralpak® AS or Chiralpak® AD.

18. A process as claimed in claim 1, wherein the mobile phase used in the chiral chromatography process is selected from:
(a) an alcohol, another organic solvent, and mixtures thereof;
(b) methanol, ethanol, propanol, isopropanol, acetonitrile, and mixtures thereof,
(c) an acetonitrile: alcohol mixture;
(d) an acetonitrile: methanol mixture;
(e) an acetonitrile: ethanol mixture;
(f) an acetonitrile: methanol mixture, wherein the acetonitrile:methanol ratio is between 70:30 and 90:10;
(g) an acetonitrile: methanol mixture, wherein the acetonitrile:methanol ratio is about 81:19;
(h) an acetonitrile: ethanol mixture, wherein the acetonitrile:ethanol ratio is between 80:20 and 95:05; or
(i) an acetonitrile: ethanol mixture, wherein the acetonitrile:ethanol ratio is about 90:10.

19.-26. (canceled)

27. A process as claimed in claim 1, wherein the mobile phase further comprises:
(a) a co-solvent;
(b) an alkylamine as a co-solvent: or
(c) diethylamine as a co-solvent.

28.-29. (canceled)

30. A process as claimed in claim 1, wherein the mobile phase used in the chiral chromatography process is recycled.

31. A process as claimed in claim 1, wherein the process is performed:
(a) at a temperature of 20-30° C.; and/or
(b) on an industrial scale.

32. (canceled)

33. A process as claimed in claim 1, wherein 1.77 kg, 10 kg, 30.7 kg or more of pramipexole is produced per day.

34.-35. (canceled)

36. A process as claimed in claim 1, wherein the yield of the pramipexole produced is 74%, 91% or more of the theoretical yield.

37. (canceled)

38. Pramipexole, or a pharmaceutically acceptable salt thereof,
(a) obtained by a process as claimed in claim 1;
(b) having a chemical purity of 99%, 99.5%, 99.83% or more as measured by HPLC: or
(c) having an optical purity of ≧96%, ≧98%, ≧99%, or ≧99.42%.

39.-45. (canceled)

46. A compound as claimed in claim 38, wherein the compound is a di-hydrochloric acid salt.

47. Pramipexole having an optical rotation of −88.7° (c−1, EtOH) or lower.

48. Pramipexole dihydrochloride having an optical rotation of −67.7° (c=1, MeOH) or lower.

49. A compound as claimed in any one of claims 38, 46, 47, or 48 for use as a medicament.

50.-51. (canceled)

52. A pharmaceutical composition comprising a compound as claimed in any one of claims 38, 46, 47, or 48 and a pharmaceutically acceptable carrier or diluent.

53. A method of treating a psychiatric or neurological disorder, comprising administering a therapeutically effective amount of pramipexole or a salt thereof as claimed in any one of claims 38, 46, 47, or 48 to a subject in need of such treatment.

54. A method as claimed in claim 53, wherein the psychiatric or neurological disorder is schizophrenia, Alzheimer's disease or Parkinson's disease.

55.-56. (canceled)

57. A method of treating a psychiatric or neurological disorder, comprising administering a therapeutically effective amount of a pharmaceutical composition as claimed in claim 52 to a subject in need of such treatment.

58. A method as claimed in claim 57, wherein the psychiatric or neurological disorder is schizophrenia, Alzheimer's disease or Parkinson's disease.
